(19) United States
(12) Patent Application Publication
(10) Pub. No.: US 2019/0115106 A1
(43) Pub. Date: Apr. 18, 2019

(54) Methods for Analysis of Digital Data

(71) Applicant: White Anvil Innovations, LLC, Needham, MA (US)

(72) Inventors: Rick A. ROGERS, Needham, MA (US); Yohann GRONDIN, Newton, MA (US)

(21) Appl. No.: 16/090,730

(22) PCT Filed: Apr. 7, 2017
(86) PCT No.: PCT/US2017/026624

§ 371 Date: Oct. 2, 2018

Related U.S. Application Data
(60) Provisional application No. 62/319,403, filed on Apr. 7, 2016.

(51) Int. Cl. G16H 50/30

(52) U.S. Cl. CPC G16H 50/30

(57) ABSTRACT

Methods for producing an enriched reference data map useful for identifying critical factors for the development of a condition of interest are disclosed. The reference data map may be used to assess the risk or likelihood of a condition of interest being realized. In the context of medicine or genetics, the methods of the invention may be used to produce a risk assessment roadmap useful for identifying elements (biomolecular constructs, biological interactions, and biological pathways) that are critical to the development of a particular disease or syndrome. The roadmap may be consulted to design treatment methods having the greatest likelihood of successfully treating or preventing the development of a disease or syndrome. Also disclosed are methods for using such a risk assessment roadmap to evaluate a specific configuration of elements for determining the changes in the configuration of elements that will result in the achievement or the avoidance of a defined condition of interest. In the context of medicine or genetics, the invention provides methods for determining the susceptibility of an individual or group of individuals to develop a particular disease or syndrome utilizing biological data of the individual or group and assessing the level of risk by referencing a risk assessment roadmap prepared according to the disclosure herein. Uncertainty in diagnosis is minimized or eliminated by these methods, and the targets, interactions, and pathways most likely to be critical for disease development, and so representing the best intervention points for treatment or prevention of the disease or syndrome, are identified.

### CLAIM OF PRIORITY

This application claims the benefit of priority to U.S. provisional application No. 62/319,403 filed Apr. 7, 2016, the contents of which are incorporated herein by reference in their entirety.

### FIELD OF THE INVENTION

This invention relates generally to biomolecular interaction analysis, mass data gathering, and mass data integration. Specifically, the invention relates to improved methods for harnessing the power of extremely large data sets, sometimes referred to as “Big Data” and exemplified by “omics” data, i.e., genomics, proteomics, metabolomics, pharmaconomics, etc., in order to identify and rank biomolecular interactions, targets, and pathways that will have the highest likelihood of controlling or determining the development of a particular disease or syndrome. From the integration of such mass data to determine relevance of biomolecular interactions, targets, or pathways to a particular disease or syndrome, an enriched reference database is produced, and such enriched reference database, representing a population or a subset of a population, can be interrogated using the genetic profile of an individual or group of individuals in order to determine susceptibility for developing the particular disease or syndrome and to identify the most effective targets for addressing such disease or syndrome therapeutically.

### BACKGROUND OF THE INVENTION

Genetic material (DNA) that makes up the chromosomes in all nucleated cells of the human body provides the complete instructions for production of all proteins in the body. Development of the field of genetic engineering and the complete sequencing of the human and many other organisms' genomes has led to a greater understanding of the inter-related function of cells and the systems that maintain life.

Along with the increased understanding of normal genetic function has come a great increase in the understanding of how variations, anomalies, and mutations in the content, configuration and operation of genetic material can result in abnormal or arrested functions or provide a genetic basis for many diseases. The genetic material of two individuals, even genetically identical twins, can vary in many ways, e.g., copy number variations in a particular gene and differences in exomes (complement of encoded proteins), CpG islands, methylation sites, coding and non-coding RNAs, and conformation of chromosomal loci, etc. All such factors can lead to differential expression of many proteins, which in some cases may lead to development of a disease or syndrome in one individual and not another.

Among the most studied and common variations within the genomes of individuals of the same species are genetic polymorphisms. “Genetic polymorphisms” refers to the presence in a population or species of two or more alleles or forms of a gene at one locus, where each allele occurs frequently enough that it is maintained in the genome of the species. The simplest genomic polymorphic variants are single nucleotide polymorphisms, or SNPs, which are variations of a single nucleotide at a given genomic locus. More complicated genetic polymorphic variants include deletion or insertion polymorphisms, for example where genetic segments are not present in one allele of a gene but are present or tandemly repeated in another allele of the same gene.

The achievement of completely sequencing the human genome and the ability to sequence any subject's entire genome within a short period of time and at reasonable cost has led to an explosion of available information regarding specific genetic polymorphic variants and in many instances their contribution, or partial contribution, to genetic disorders or the development of a disease. Genetic polymorphisms may be silent, meaning that the variant leads to no detectable effect on gene expression or function, or active, wherein the variation leads to differential transcription or expression of the gene or alters the nature of an expressed protein encoded by the gene. For example, a SNP located in an exon of DNA encoding a protein may lead to the expression of a protein of a different amino acid sequence or a splice variant of the protein, or may even arrest expression of the protein if the SNP leads to the creation of a stop codon at that locus. A SNP in an intron may also affect gene expression, e.g., by altering mRNA splicing, interacting with gene transcription products, or interacting with cellular machinery. SNPs in non-coding transcriptional regulatory regions may diminish, arrest, or amplify gene expression.

It is estimated that there are more than five million SNPs in the human genome with a frequency of 10% or greater. Since each SNP or group of SNPs reflects a single ancient mutation event in an ancestral chromosome which has been propagated in succeeding generations of progeny, SNPs are useful in population genetics to study family or subpopulation origins, and in forensic science to identify individuals or establish blood relationships. SNPs and other genetic polymorphisms may also become markers associated with risk of developing diseases or syndromes.

There are several human diseases where development of the disease is highly correlated to a genetic polymorphism in a single gene. Cystic fibrosis, for example, is caused by conformational changes in the cystic fibrosis transmembrane conductance regulator (CFTR), which changes can result from a single genetic mutation altering one amino acid, the most common of which is the deletion of phenylalanine at position 508 (Δ508F) of the CFTR protein. See, Davies et al., Proc. Am. Thor. Soc., 7: 408-414 (2010). In another example, the incidence of females who exhibit a mutant form of either breast cancer predisposing gene BRCA1 or BRCA2 going on to develop early-onset breast cancer is high enough that the presence of BRCA1 or BRCA2 mutations alone has become a determinative risk factor triggering increased monitoring or preventive therapeutic intervention, even in individuals who are asymptomatic for cancer. See, e.g., U.S. Pat. Nos. 5,693,473 and 5,837,492. Other diseases or syndromes for which a single SNP or polymorphic variant is considered sufficient for diagnosis include community acquired pneumonia (SNP in TNFβ gene), depression (SNP in A-Kinase Anchor Protein 9 gene), deep vein thrombosis (SNPs in coagulation factor F5 gene), Alzheimer's disease (SNPs in apolipoprotein E gene), polycystic kidney disease (SNP in PKD1 gene or PKD2 gene), and coronary artery disease (SNP in GCH1 gene). U.S. Pat. Nos. 6,383,757; 7,794,933; 8,771,946; US 2011/0200994.

In spite of many observed high correlations between monogenic variants and development of disease, the etiology of most human diseases (including most of those mentioned above) is not a monogenic affair but involves the participation of multiple genes and gene products which are interrelated functionally and manifested within biochemical pathways, spatial orientation within cells, 3-dimensional tertiary structures, and the positioning of molecules relative to each other. For example, on average a given protein typically interacts with 6 to 20 other proteins, and in some cases many more, into the hundreds. This makes analysis to pinpoint the causative agents in disease to a level of complexity that defies systematic analysis and depends on trial and error, or hypothesis driven research applied to single features of particular experimental interest. A limitation for computational analysis of genetic material is commonly encountered when introns and exons are subjected to computational analytical processing. Typically, there are more introns present in a given DNA sequence than exons, thus limiting pairwise comparisons necessary in computer processing because the data are “unbalanced”. The present day state-of-the-art genetic material analysis does not consider global or composite considerations as a result of these pairwise constraints.

Assessment of risk for developing a disease by detection of only a single or limited number of genetic polymorphic variants may lead to unnecessary treatments, to treatments that prove to be ineffective because they address irrelevant symptoms rather than the true cause of the disease, or to treatments that are blind in that targets for effective therapeutic intervention are overlooked or undetected by the diagnostic assessments that are followed. The example of BRCA1 diagnosis of breast cancer risk furnishes an illustration of the uncertainty inherent in basing a diagnosis of a disease as serious as breast cancer on the presence or absence of a mutation in a single gene, where development of the disease obviously involves a host of genetic factors. The incidence of those having BRCA1 mutations developing breast cancer is not 100%, rather only about 45% of early onset breast cancer patients show a BRCA1 mutation. (See, U.S. Pat. No. 5,693,473.) Despite the fact that 60% of individuals harboring the mutation would not proceed to develop breast cancer, BRCA1 mutation is considered an appropriate biomarker of disease sufficient to trigger oncological intervention. The downside risk of ignoring the BRCA1 mutation risk factor is sufficiently fearsome that many patients and their oncologists opt for treatment on the basis of the discovery of a BRCA1 mutation alone. If more of the factors leading to breast cancer were known and considered, improved treatments or more accurate (less uncertain) assessment of the true risk of developing the disease could be made. The present invention addresses this failure of diagnostic methodology in a robust, unbiased, systematic manner.

These diagnostic shortfalls or errors occur and are occurring in the midst of a superabundance of genetic data growing out of the sequencing of the entire human genome and the tabulation of huge amounts of data on protein activities, protein-protein interactions, and the metabolism of proteins and other chemical entities in vivo.

Accordingly, there is a need to develop methods to increase the accuracy of diagnostic assessments drawn from genetic information and a need to bring the power of large amounts of data (i.e., “Big Data”) to bear on the assessment of the susceptibility of individuals or groups of individuals at risk to develop a particular disease or syndrome. More accurate assessment of disease risk and a clearer, more comprehensive identification of targets for therapeutic intervention in the development of a disease or syndrome are the goals of the present invention. The present invention provides a means to discern relatedness among biological factors contributing to disease and to capture biological meaning from reported aspects of function and structure of individual biomolecular constructs.

### SUMMARY OF THE INVENTION

The present invention relates to methods for analysis of omics data to discover the critical biological interactions relevant to health and disease. The methods minimize or eliminate uncertainty from identification of the main contributors to development of a disease or syndrome. The refined reference dataset of biological interaction networks relevant to a particular disease or condition can be interrogated with individual genetic or biomolecular profile information to accurately determine the susceptibility risk of a person for developing a particular disease or condition. The reference dataset can also be used to guide patients and physicians to the most effective treatments for therapeutic intervention in the development of a disease or condition in the patient.

Prior to the methods described herein, the state of the art in the field of genetic analysis relied on levels of statistical significance visualized, for example, by a Manhattan plot. Although such analyses provided highly accurate assessment of genetic differences from a population, they do not relate to functional interpretation of the proteins corresponding to the genes represented in the Manhattan plot. The concept provided by the present invention is not sensitive to the point-by-point analysis provided by the Manhattan plot, or related analyses, which has become the standard metric by which genetic variations are analyzed. The present methods begin where the value of the Manhattan plot ends, by analyzing data points in terms of their inter-relatedness or interactions.

The method of the present invention may be used in an initial phase to establish a fully integrated, multidimensional map of biomolecular constructs, their interactions and associations, the map giving accurate information concerning the risk associated with a given physiological condition. This map is a risk assessment tool that is derived using mass data sources, commonly referred to as “Big Data” or “omics” data, such as genomics data, proteomics data, metabolomics data, pharmaconomics data, etc. According to the invention, these mass data are treated utilizing theory to derive a robust solution from a multifaceted analysis, as an alternative approach to the typical hypothesis-driven experimentation, which proceeds by testing and analysis of single independent markers that are usually phenotypically, phenomenologically, or clinically defined.

After establishment of a risk assessment map or tool, a practitioner may proceed with an application phase, which interrogates the risk assessment map using individual profile data, derived, e.g., from a biological sample, for assessment of individual risk to develop the tested physiological condition. The invention thus enables interpolation of individual risk to develop the tested physiological condition from a complex biomolecular profile, unique to the individual, but with enough commonality to associate with a mapped physiological condition defined by theoretical, network-applied metrics.

In the field of medicine, an embodiment of the present invention provides (I) a method for producing a risk assessment map for a selected physiological condition to be diagnosed or treated (physiological condition of interest) and (II) a method for determining the susceptibility or risk of an individual or group of individuals for developing the physiological condition. The initial phase (I) of such an embodiment is a method for producing a risk assessment map comprising the steps:
(a) selecting a set of biomolecular constructs associated with a physiological condition to be diagnosed or treated;
(b) constructing an integrated multidimensional network detailing biophysical and biochemical properties and interactions of the selected biomolecular constructs;
(c) tuning the amount of information to be retained in the multidimensional network using mathematical functions to ensure maximization of the information content, minimization of bias, and reduction of uncertainty; and
(d) computing the criticality of each biomolecular construct in the resulting map using structural and functional metrics derived from mathematical graph theory, statistical physics, and systems biology.

Biomolecular constructs that may be selected in step (a) can be any biophysical entity capable of having a physical, chemical, or metabolic effect on or association with the physiological condition of interest. Such biomolecular constructs include, for example, genetic polymorphisms (e.g., single nucleotide polymorphisms or SNPs), genes, proteins, protein complexes, etc., which, for the purposes of this invention, are recorded in mass data collections (mass databases, omics data). The data used to construct the integrated informational network of step (b) includes biochemical, structural, and functional information related to each element of the set of biomolecular constructs identified in the previous step (a), together with information regarding interactions of each element with other biomolecular constructs retrievable from one or more mass data collections. Information retrieval is repeated for every interacting biomolecular construct from all data sources, then integrated to the set of biomolecular constructs until the system percolates. A system is said to percolate when there has been at least one biological interconnection or pathway established between any two elements of the initial data collection (a). This results in an integrated multidimensional network. The tuning in step (c) of the information in the multidimensional network resulting from step (b) uses maximization of entropy in a technique adapted from statistical physics and applications in other fields such as autofocusing in photography and microscopy and gravitational lensing from astrophysics. Maximization of entropy in the multidimensional network eliminates data having minimal relevance to the physiological condition of interest and thereby eliminates bias from the network. Application of further metrics in step (d) results in a risk assessment map that can be used in a further phase to calculate the risk of individuals to develop the physiological condition of interest.

The second phase (II) of the embodiment is a method for determining the susceptibility of an individual to develop the physiological condition of interest comprising the steps:
(a) establishing a profile for an individual by identifying the subset of biomolecular constructs corresponding to the set selected in the phase I method from a biological sample obtained from the individual;
(b) computing the risk of the individual to develop the physiological condition of interest by mapping the profile of step (a) to the risk assessment map obtained in phase I.

This invention provides a means to identify the main contributors to a disease or syndrome and a means for predicting susceptibility to developing such disease or syndrome. The contributing factors are genes, gene products, and their interactions that are derived from lists of candidate biomolecular constructs and which are identified through the construction of unbiased, multidimensional data networks of biomolecular construct interactions. The analysis techniques of the present invention can be applied to a variety of technical fields, including personalized medicine, aging, predictive medicine, therapeutic intervention, risk analysis, epigenetic change resulting from environmental exposure, etc.

The method of the present invention identifies the risk of an individual to develop any one or a number of physiological conditions that results from cellular dysfunction triggered by changes or abnormalities in multiple biochemical elements, such as DNA, proteins, metabolic processes, etc. The invention involves: (1) the construction of a multidimensional biomolecular map capturing essential features of the tested physiological condition (physiological condition of interest); and (2) the determination of the risk contribution of each element of the map to the tested physiological condition. The methods of this invention incorporate principles of data analysis from disparate technical fields such as microscopy (autofocus), astrophysics (gravitational lensing), biochemistry (biomolecular interactions), mathematics (graph theory), information theory (networks), engineering (risk analysis), physics (entropy) and systems biology (biological data integration and modelling). Data analysis methods derived from these fields have been unified under the general realm of statistical physics. This invention has been reduced to practice by employing custom-designed algorithms that sieve through “omics” databases to capture the biochemical information critical to the mapping and risk calculation processes. The algorithms used in the steps of the methods described herein are designed to render biological mass data into values that can be exploited by the computational, mathematical, biophysics and physics concepts used in the methods, in much the same way that chemical reactions are often expressed using a defined equation-based rule set. For example, an algorithm described below allows the practitioner to calculate the entropy—a thermodynamic quantity—from a network of protein-protein interactions, to eliminate bias from the analysis of a massive dataset. The refined dataset resulting from performing the method of this invention bears no resemblance to conventional diagnostic prediction methods, which only determine the risk to an individual based on pair-wise comparisons, making one or a series of tests on independent markers or indicators having an observed correlation to a particular physiological condition of interest.

In another embodiment, in the field of medicine, the invention is useful to compute the risk of developing a particular disease using a biological sample obtained, e.g., from saliva, blood or other biologically relevant source. The biological sample is processed to tabulate biomolecular constructs such as DNA/RNA, exons, introns, single strand breaks, SNPs, etc., using standard genomic sequencing technologies. Output from the sequencing is then used in a refinement process to determine the profile of genetic variants. The invention is implemented in accordance to the specificity of the particular disease of interest using biomolecular data. For example, the process may consider genetic variants, such as mutations or single nucleotide polymorphisms, as an input. A recursive or iterative process is used to retrieve data from mass data sources (“omics” data) associated to the input. These include, but not are limited to, protein-protein interactions, cell-type-dependent expression, metabolic-protein interactions, functional domain definition data, for example. Application of a series of data analysis functions, i.e., modified auto-focusing algorithms, Shannon's entropy, and gravitational lensing approaches, govern the amount of data retrieved, the extent of the processing, and the quality of the multidimensional map that results from the application of the applied functions. Quantitative graph metrics such as clustering, betweenness, assortativity, are then applied to the map, to determine the association of each element of the map with its functional domain, relationship to other elements, and criticality in the system.

The invention uses a stepwise progression with various combinations of process and complex mathematical equations to calculate risk associated with candidate genes and gene products to compute total risk for an individual. An advantage of the present invention is that the method is insensitive to the emphasis on pairwise comparisons that are common to other genetic analysis tools. A series of algorithms parse information to calculate risk based on a given profile. Information derived from networks of interactions at the steepest rate of change or interaction of all known proteins is utilized. Information contained in the manner in which these proteins interact with each other is treated using a series of quantitative metrics, based on graph theory and mathematics, to calculate the risk for developing a particular disease associated with the candidate genes or gene products.

In embodiments, the technology of the present invention can be used to compute the risk of development for a disease state under clinical investigation, provide a risk score for an individual or group of individuals, and reveal the potential treatments, including alternate treatment options. The present invention also provides predictive outcome for developing a condition or susceptibility to a particular condition.

### BRIEF DESCRIPTION OF THE DRAWINGS

FIG. 1 is a flow chart diagram showing the steps involved in creating a risk assessment dataset for a particular disease or condition, based on biostatistical analysis of genetic polymorphism data and omics data concerning the polymorphism-implicated proteins, their activities, and interactions with other proteins. The diagram also shows the steps for interrogating the risk assessment dataset with genetic profile information from an individual or group of individuals to ascertain risk of development of the disease or condition and to identify the most effective targets for therapeutic intervention in the disease or condition.

FIG. 2 is a diagram of a hypothetical protein interaction network considering five proteins, A, B, C, D, and E. The lines connecting proteins indicate a reported or expected protein-protein interaction between two proteins. From this group of proteins, protein A is regarded as having a first-degree interaction with protein B, and second-degree interactions with proteins C and D. Protein A also is considered to have a third-degree interaction with protein D. Proteins A-D form an interaction network; protein E does not have any known or expected interactions with any of the other proteins (in this group).

FIG. 3 shows the increased complexity of the matrix map, created using the protein interaction data for fifty randomly selected proteins in Arteriosclerosis Adjacency Matrix/Data Set 4 described in Example I.

FIG. 4 shows a matrix map created using the protein interaction data for two hundred selected proteins in Arteriosclerosis Adjacency Matrix/Data Set 4 described in Example I.

FIG. 5 shows a map created using the protein interaction data for 574 proteins in the Arteriosclerosis Adjacency Matrix/Data Set 4 described in Example I.

FIG. 6 shows a plot of the maximization of function Q from the Arteriosclerosis Adjacency Matrix/Data Set 4 described in Example I.

FIG. 7 is a flow diagram showing the steps of a method according to the invention as illustrated in Examples I and II, for assessing risk of an individual for developing, e.g., arteriosclerosis. The flow diagram shows the steps involved in making a risk assessment map (Phase I) that can be used in a further Phase II to calculate the risk (susceptibility) of individuals to develop the disease condition.

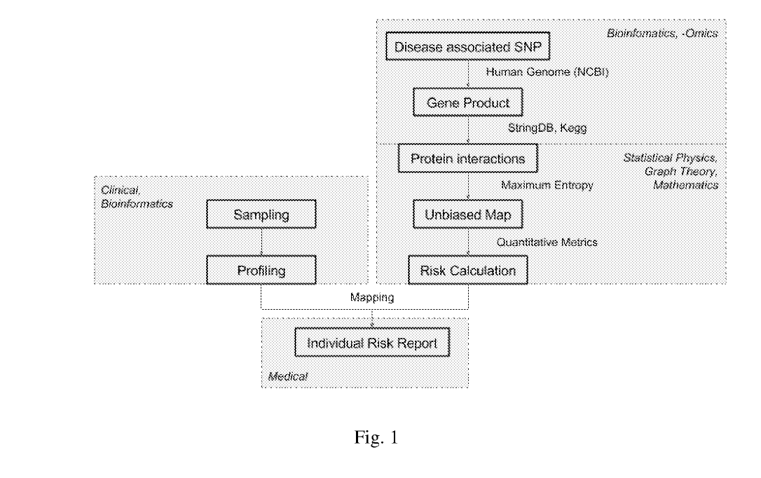

### DETAILED DESCRIPTION OF PREFERRED EMBODIMENTS

The present invention is directed to analytical methods to provide risk assessment tools for identifying and ranking the genetic products and interactions that are critical in the development of a disease or biological condition. A reference dataset of critical biomolecular targets, interactions, and pathways relevant to development of a particular disease or condition may be produced, and such refined reference dataset may be interrogated with genetic profile information of an individual or group of individuals to determine risk of developing the disease or condition and to assist in devising an effective approach to diagnosis, treatment or prevention of the disease or condition.

In order to more clearly describe the present invention, the following terms and definitions will apply:

The terms “mass data”, “massive data”, “mass data collection”, “mass database”, and “mass dataset” are used interchangeably and refer to any repository of data or information relating to a very large number of elements. As a practical matter, a mass data collection or mass database will retain in one repository information relating to at least 1000 elements, for example a database containing information on 1000 or more different proteins may be regarded as a mass data collection or mass database for the purposes herein. Mass data collections that seek to be a central repository for information on the entirety of a category of elements will often be referred to herein as “omics data”, in that information pertaining to an entire -ome or universe of elements is collected. For example, a data repository designed to hold information about all known proteins, otherwise known as the proteome, is referred to as proteomics data; likewise, information pertaining to all known genes, otherwise known as the genome, is referred to as genomics data. Other examples of omics data include, metabolomics data (data pertaining to the totality to metabolic processes), pharmaconomics data (data pertaining to the totality of pharmacologic compounds and substances), and bacteriomics data (data pertaining to the entirety of bacteria, e.g., in a given environment, as in, e.g., the gut bacteriome, describing all species of bacteria found in the gut). The present invention provides a useful way of extracting critical information pertaining to a given condition from omics data.

The term “biomolecular construct” is used herein to describe any chemical or molecular entity (natural, manufactured, or engineered) that relates to a biological property, function, or system. A biomolecular construct may be a gene, a gene product (protein), isolated nucleic acid molecules (coding DNA/RNA, non-coding DNA/RNA, micro RNA, complementary sequences, aptamers, etc.), organic compounds, metabolites, peptides, haptens, co-factors, enzymatic substrates, and the like. In short, the term “biomolecular construct” is intended to be a universal term for the elements participating in any chemical, biochemical, physiologic or biological process on which data is collected.

The terms “data map”, “risk map”, and “data roadmap” as used herein are interchangeable terms referring to a refined data product of a method according to the invention that identifies critical elements and element interactions relevant to a tested condition. In medical applications, the elements are genes, gene products (proteins), and protein interactions, and the tested condition is a disease or syndrome dependent on the presence or absence of one or more proteins or protein interactions. In genetic testing applications, the elements identified in a data map according to the invention are genes and clusters of genes, and the tested condition is a genetic disease or syndrome dependent on the presence or absence of a functional gene or multiple genes.

As used herein, a “tested condition” or “condition of interest” refers to any state or phenomenon that may result from the cumulative effect of one or more elements on which mass quantities of data are collected. An example of a condition of interest in the field of medicine or genetics would be a disease or disorder that is the result of the presence or absence of one or more biomolecular constructs or interactions between biomolecular constructs, and the biomolecular constructs would be the elements, such as genes, gene products, protein-protein interactions, and metabolic pathways, on which mass amounts of physical and structural data are collected.

A “multidimensional network” refers to a data collection identifying not only elements but interactions and dependencies between elements. The interactions may be functional, structural, or temporal.

The present invention provides a method for processing mass data collections with respect to a condition of interest to produce a refined data map of critical data elements and element interactions having an impact on the condition of interest. The resultant data map is useful as a tool to accurately assess the risk of the condition of interest arising or developing under a given set of conditions. The data map is also useful as a guide to points of intervention that are critical in the development of the condition of interest, which may in turn be used to devise ways to prevent or ameliorate the condition of interest.

In its most basic aspect, the process for production of a data map according to this invention proceeds by the following steps:
(a) selecting from a mass data collection a set of data elements having an association with a condition of interest;
(b) constructing an integrated multidimensional network from the initial selected set of data elements by collecting data, for each element, relating to interactions with any other element;
(c) sorting the information from the multidimensional network using mathematical functions to eliminate information of lesser relevance to the condition of interest, to ensure maximization of the retained information content, minimization of bias, and reduction of uncertainty; and
(d) applying quantitative metrics to the retained information of the multidimensional network to create a data map that gives relative weight to the retained elements and element interactions, identifying the criticality of each element and interaction with respect to the condition of interest.
   The data map that results from this process provides a tool for identifying the pattern of elements that brings about the condition of interest. By comparison of a given set of elements and interactions against the data map, the likelihood of the condition of interest coming to realization can be assessed. For a desirable condition of interest, the changes relating to the elements and their interaction pathways that are necessary to achieving the condition of interest may be identified; for an undesirable condition of interest, such as a disease, comparison of the given set of elements and interactions with the data map identifies the critical elements and interaction pathways to be changed or blocked so as to avoid the development of the condition of interest. The applications for the method that are most immediately apparent are in the fields of medicine and genetic testing, but the mass data analysis methods described herein can be applied to any field where the elements of critical importance to the development of a condition of interest must be identified, either for successful achievement of the condition or timely prevention of the condition.

In medical applications, a data roadmap resulting from practicing the invention identifies the critical biomolecular constructs (i.e., protein or genetic elements, protein interactions, and metabolic pathways connecting protein elements) that are critical to the development of a tested disease condition or syndrome, and thus provides a tool for assessing the risk of an individual or group of individuals to develop a disease condition or syndrome, such as cancer, autism, hypertension, arteriosclerosis, osteoporosis, mental illness, dementia, various forms of blindness, and a wide variety of diseases and syndromes that result from multigenetic interactions. In the field of genetic testing, a data roadmap resulting from practicing the invention identifies the critical genetic elements and interactions between genetic elements critical to the development of a genetic trait or a genetic condition or syndrome, which in turn provides a means for assessing the risk of an individual or group of individuals (such as a family, a tribe, a group of individuals subjected to common epigenetic factors) for developing a genetic trait or a condition or syndrome resulting from multigenetic factors.

The invention will be described in more detail below with reference to applications in the fields of genetics and medicine, where “omics” data are available for analysis of biophysical conditions of interest. However, it will be appreciated by those skilled in any field where mass data collections (e.g., so-called Big Data) are available for processing to analyze the development of a condition of interest, that the present invention is likewise applicable to provide a means of rendering mass data, to identify the data elements and element interactions of critical importance to development of the condition of interest. It is noted that phenomena resulting from the effect of one or more elements for which there are little or no available data may not be advantageously analyzed according to this invention, since too little information would exist to accurately distinguish between elements and interactions that are critical and those that are of negligible relevance to a tested condition: critical elements would be eliminated from the final data product or non-critical elements would be retained, confounding the advantages obtainable by this invention. In such barren data environments, traditional hypothesis-driven research investigating single elements at a time is at least as advantageous as practicing the presently described methods.

### Mass Data Collections

The present invention relies on the processing of massive quantities of data available in mass data collections (mass databases or data repositories) as an alternative to the hypothesis-driven, step-wise investigation of single data elements such as individual biological markers. Construction of a risk map in the medical/genetics field requires a large and varied amount of biological data, and for a wide variety of conditions that may be of interest to researchers, medical practitioners, and genetic advisers, a wealth of collected biological data exist, including data pertaining to but not limited to gene and protein structure, protein-protein interactions, cell-dependent gene and protein expression, gene activation, variable gene expression, genetic polymorphisms (such as single nucleotide polymorphisms), genetic mutations, protein isoforms, etc. Such data are collected and available in public and private (subscription) repositories and can be accessed and analyzed by computer, e.g., over the internet. Some of the most frequently interrogated mass data sources are discussed below.

GWAS Catalog (http://www.ebi.ac.uk/gwas)
The Genome-Wide Association Studies (or GWAS) Catalog, is a database collecting genotyping and analysis data on >100,000 SNPs without regard to gene locus or gene content, from published peer-reviewed medical and scientific journal articles and science news reports. The GWAS Catalog is co-curated by the National Human Genome Research Institute (NHGRI) of the National Institutes of Health (NIH) and the European Molecular Biology Laboratory-European Bioinformatics Institute (EMBL-EBI). It is accessible online at http://www.ebi.ac.uk/gwas. This database contains information on published GWAS studies, giving 33 fields of information for each study, including the name of the study, sample size, SNP, mapped position, chromosome location, p-value, odds ratio, etc. This database is not exhaustive and extracted information may need to be supplemented by consulting other sources.
SNPedia (http://www.snpedia.com/index.php/SNPedia)
This database provides a high level summary of SNP-centric published information. Data provided include disease-association risk, subpopulation frequency, published GWAS data such as p-values, odds-ratio, etc.
STRING Database (http://string.embl.de/)
The STRING database of protein-protein interactions is curated by the Swiss Institute of Bioinformatics (SIB), the Novo Nordisk Foundation Center for Protein Research (CPR), and the European Molecular Biology Laboratory (EMBL). STRING is a database of known and predicted protein interactions including direct (physical) and indirect (functional) associations, derived from four sources—genomic context, high-throughput experiments, conserved co-expression, and interactions reported in the scientific literature. The current version of the STRING database (no. 10) includes interaction data covering 9.64 million proteins from over 2000 organisms. The database is located at http://string-db.org. The STRING information is parsed in several files. A line entry gives a set of two interacting proteins, each labeled with a unique ENSP number, for example 9606.ENSP00000261637 (9606 refers to human proteins; this particular ENSP number designates UTP20 (a.k.a. DRIM), a component of the U3 small nucleolar RNA protein complex). A STRING line entry also includes eight additional fields (i.e., neighborhood, fusion, co-occurrence, co-expression, experimental, database, text mining, and combined score), which contain confidence-level scores assigned by the database curators based on the nature of the interaction of the two proteins as derived from the data sources. In the examples that follow, these additional fields were not mined, and what was utilized was only the fact of the protein-protein interaction pairing of the Primary Protein and the Interacting Protein from this database.
KEGG Metabolic Pathway Database (http://www.genome.jp/kegg/)
The KEGG (Kyoto Encyclopedia of Genes and Genomes) database of genetic and molecular pathways integrates genomic, chemical and systemic functional information. Catalogs of genes from fully sequenced genomes are linked to systemic functions of the cell, the organism and the ecosystem. See, Kanehisa, M., “Toward pathway engineering: a new database of genetic and molecular pathways,” Science & Technology Japan, 59:34-38 (1996). The KEGG database resource is curated by Kanehisa Laboratories and can be accessed at http://www.genome.jp/kegg.
Human Protein Atlas (http://www.proteinatlas.org)
The Human Protein Atlas contains information for a large majority of all human protein-coding genes regarding the expression and localization of the corresponding proteins based on both RNA and protein data. The atlas consists of four subparts; normal tissue, cancer, subcellular and cell lines with each subpart containing images and data based on antibody-based proteomics and transcriptomics. Version 14 of the Human Protein Atlas contains RNA data for 99.9% and protein data for 86% of the predictive human genes and includes more than 11 million images with primary data from immunohistochemistry and immunofluorescence. The Human Protein Atlas is a project funded by the Knut and Alice Wallenberg Foundation. It is a publicly available database, accessible at http://www.proteinatlas.org. The main sites are located at Alballova and SciLifeLab, KTH—Royal Institute of Technology, Stockholm, Sweden, and the Rudbeck Laboratory, Uppsala University, Uppsala, Sweden.

### Human Genome

The human and 1000 other genomes are available from the National Center for Biological Information (NCBI), a division of the National Library of Medicine (NLM) at the National Institutes of Health (NIH). The publicly accessible website at www.ncbi.nlm.nih.gov is a repository for a collection of searchable databases pertaining to all aspects of genetics and medicine. Databases collecting data on DNA, RNA, genes and expression, genetics and medicine, genome maps, gene homology, genetic variants including SNPs, proteins, sequence analysis, taxonomy, chemicals and bioassays, and others are available, as well as software and tools for conducting searches and analysis of data.

### Scientific Literature

Online libraries of published research (e.g., MEDLINE, EMBASE, etc.) may also be searched to compile focused data collections to supplement and update the other mass data repositories.

### Creation of an Integrated Multidimensional Network

Using data extracted from mass data collections such as those discussed above, retrieved on the basis of an association with a condition of interest, an integrated network is composed of biomolecular constructs that interact structurally and functionally with each other. To construct this network, candidate gene products (pertaining to a tested condition) are placed in a restricted network, based on interactions between these proteins retrieved from mass databases that contain information available from research, clinical studies, and literature reports. Interactions may be about the genomic, metabolic, biochemical, structural, and other proteomic aspects of proteins of interest. Each protein's interactions with all other proteins are investigated, one protein at a time, until all reported interactions for all the proteins are collated. The resultant multidimensional network of proteins is then tuned to reveal important associations and pathways having the most relevance to the tested condition.

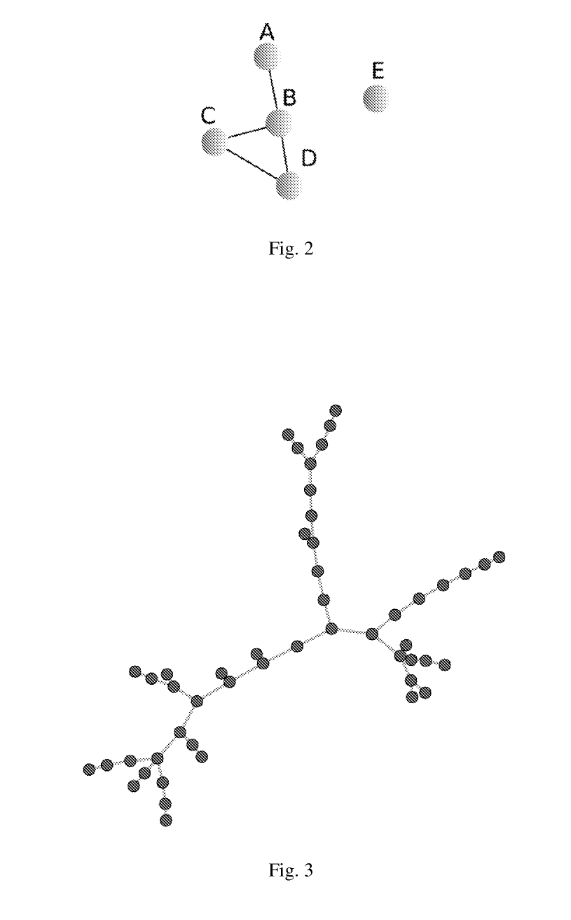

The creation of a multidimensional network for five proteins (A-E) retrieved from a mass data collection is illustrated in FIG. 2. Initially the five biomolecular constructs (in this illustration, five proteins), A, B, C, D, and E, are retrieved to form an initial set on the basis of some tested condition, for instance, an association of each protein with arteriosclerosis (see Example I, below). Mass data sources having information on biological interactions between proteins are interrogated to create a network of protein interactions, with the interactions illustrated in FIG. 2 by lines connecting the proteins A, B, C, and D. Each interaction may be genomic, metabolic, biochemical, functional, or any other type of association reported for two individual proteins in the scientific literature or through experimentation. This is what makes the network multidimensional. In FIG. 2, protein B is seen to have reported interactions with proteins A, C, and D. When each of the proteins in turn has been analyzed for interactions, and no additional interactions are found in the data sources, the network is complete. In the data set illustrated in FIG. 2, the protein E has no reported interactions with any other protein of the set. Protein A is found to have a first degree interaction with protein B, a second degree interaction with protein C, and a second degree and a third degree interaction with protein D.

### Tuning the Protein Interaction Network to Eliminate Bias

The interaction network created from the initial biomolecular construct data set contains a wealth of information, but it may be regarded as highly overinclusive with respect to the tested condition. Treatment of the network data to eliminate less reliable or less important data in order to maximize the reliability of the data is necessary.

This tuning of the network is carried out by applying principles from other disciplines, such as autofocusing and gravitational lensing. Application of these otherwise unrelated disciplines allows the practitioner to maintain a high degree of flexibility and versatility in the nature of the interactions used, while capturing a large amount of meaningful information concerning any two elements e.g., proteins.

Interactions between proteins can be physical, such as the binding of proteins within a protein complex, or they can be functional, such as the co-expression of two proteins under given conditions. The elements of data used to generate the interaction network is iteratively adjusted to find the point that generates a network with highest information content of biological interactions. The maximal information focal point is defined by the function, S, of formula (1):

$$S = - {\sum }_{r} {p}_{r} log {p}_{r} \qquad ( 1 )$$

where pᵣ is the probability of a discrete value xᵣ, for example the degree of interactivity of a vertex in the network. Supposing constraint, C(pᵣ), is applied to the network—for example the homeostatic state of a cell defined by its energy metabolism and microenvironment—the maximization of (1) subject to constraint C(pᵣ) ensures the generation of a network that agrees with the known information while avoiding bias on the missing information. This method is an application of the maximum entropy principle, modified to generate a network of biological interactions that can be exploited to assess risk associated to a patient. To minimize bias and uncertainty requires both the use of information theory and statistical physics to refine the massive amounts of data being processed.

The maximum entropy method is used in various fields to reconstruct images from imperfect or insufficient data. For example, this method reconstructs images of distant objects in astronomy using gravitational lensing or in the field of microscopy where deconvolution is used to deconvolve out-of-focus, sub-resolution features into sharp, well-defined contrast. See, Buck, B., & Macaulay, V. A., Maximum entropy in action: A collection of expository essays, (Oxford: Clarendon Press, 1991). A simple fitting process, for example, would lead to many possible solutions and leave the problem of deciding which one is the correct one. Maximizing the entropy ensures that the reconstructed image is the most probable image given the data. The lack of complete data is commonplace in biomolecular construct interaction networks, with the identical problem of discriminating between the many solutions that fit the available data. The maximum entropy method is used to reconstruct the multidimensional network, akin to the reconstructed image obtained using gravitational lensing.

A key feature of this invention is the ability to identify the most useful data in an unbiased way, by calculating the contribution to entropy made by each of the interactions comprising the network dataset. Considering the entropy calculations serially, a plateau is reached indicating the data subset of interaction networks exhibiting maximum entropy. Once the plateau is reached, further refinement to identify datasets of high entropy is possible, but the gain in entropy is no longer so significant as to justify the effort. Stated another way, once the removal of bias from the starting network dataset reaches a satisfactory degree, further reduction of bias is not informative. Treating the dataset to maximize entropy is a means of extracting data from the dataset without bias, yielding a collection of the most useful data.

### Application of Quantitative Metrics

Additional metrics are applied to the unbiased multidimensional network of interactions of data set elements (e.g., biomolecular constructs). For protein interaction networks, for example, structural and functional properties are often interconnected, so that changes in structural parameters may affect function and vice versa. Structural parameters include, but are not limited to, degree of connectivity, clustering coefficient, assortativity, centrality, diameter, etc. Functional parameters include, but are not limited to, turnover rate, metabolic efficiency, gene activity, etc. The unbiased data need to be weighted to identify biomolecular constructs, interactions, and pathways that are critical to the tested condition. Graph metrics are applied to define the point of focus for the data set.

This is another example of adoption of principles from another technical pursuit. Graph metrics is an approach used to conduct autofocus on microscopes and digital cameras. One of the techniques, based on contrast detection, consists in maximizing the difference in intensity between adjacent pixels in a two-dimensional field. In microscopy, this is done by moving the stage or objective up or down until maximal contrast is achieved, ensuring the maximum return of information. This technique relates to a two-dimensional system where pixels have only 2 horizontal and 2 vertical neighbors. To account for the multidimensionality of the reconstructed multidimensional network, several graph metrics are used instead of contrast detection. A graph metric is a calculated value that characterizes one of the structural or functional properties of a graph or network. Structure and function of biomolecular constructs are interconnected, therefore changes in structural parameters may affect function and vice versa.

Useful graph metrics include, but are not limited to, degree of connectivity (discussed supra, corresponding to first degree interactions between proteins), degree of clustering, assortativity, and graph diameter. To develop an accurate risk assessment map, principals of connectivity, clustering, and betweenness are applied to the data in order to produce a more accurate result. Omitting any one of these metrics is likely to lead to a less accurate result, although the resultant data set would still have improved accuracy and utility over the mass data sets initially interrogated or the refined data set obtained by maximizing entropy alone. Additional metrics are contemplated and are likely to improve the accuracy of the end result. Such metrics include, e.g., centrality (clustering coefficient/diameter), betweenness, β-complexity (see, e.g., Raine, D. J., et al., “Networks as constrained thermodynamic systems,” Comptes Rendus Biologies, 326(1):65-74 (2003)), and the like.

### Degree of Clustering

The degree of clustering of a network is a statistical measure that provides information on the interconnectivity of neighboring nodes. It is given by the clustering coefficient, C, which is the average over the network of the clustering coefficient of each of the nodes (Watts, D. J. and Strogatz, S. H., “Collective dynamics of ‘small-world’ networks,” Nature 393(6684):440-442 (1998)). The clustering coefficient, Cᵢ, of node i is calculated as the ratio of the number of links between nodes connected to i, to the number of possible links between all those nodes connected to node i. The number of triangles at node i is obtained from the diagonal element—counted twice—of the cubed adjacency matrix of the network. The number of possible triangles is given by kᵢ (kᵢ−1)÷2. The clustering coefficient of the whole network is then

$$C = {N}^{- 1} {\sum }_{i} \frac{{a}_{ii} [ 3 ]}{{k}_{i} ( {k}_{i} - 1 )} ,$$

where kᵢ is the degree of connectivity of node i, aᵢᵢ is an element on the diagonal of the adjacency matrix A that corresponds to the network, and N is the number of rows (i) and columns (i) in the network, so that N×N is the total number of elements in the matrix. An adjacency matrix, A, mathematically represents a network where the intersection at each column position and each row position represents the interaction between two biomolecular constructs (e.g., a gene, a gene product, or a metabolite, etc.).

### Assortativity

Assortativity defines the preference for nodes of a given degree of connectivity to associate with each other. It is measured by the assortative coefficient, r. To define r, let eᵢⱼ be the joint probability distribution of the degrees of the nodes at the ends of a randomly chosen link, not counting this link itself in the nodal degrees (Callaway, D., et al., “Are randomly grown graphs really random?”, Physical Review E: Statistical, Nonlinear, and Soft Matter Physics, 64(4):041902 (2001)). Then r, (−1≤r≤1), is given by

$$r ≡ \frac{{\sum }_{ij} ij ( {e}_{ij} - {q}_{i} {q}_{j} )}{( {\sum }_{k} {k}^{2} {q}_{k} - {( {\sum }_{k} {kq}_{k} )}^{2} )}$$

where the normalized ‘remaining degree’ distribution (Callaway, D., et al., “Network robustness and fragility: percolation on random graphs,” Physical Review Letters, 85(25):5468-5471 (2000), Barabasi, A. L. and Albert, R., “Emergence of scaling in random networks,” Science, 286(5439):509-512 (1999)), qₖ, is

$${q}_{k} = \frac{( k + 1 ) {p}_{k + 1}}{{\sum }_{j} {jp}_{j}}$$

The coefficient r is positive for assortative networks and negative for disassortative ones. It has been measured that sociological networks are assortative, that is, nodes of large degrees of connectivity are preferentially connected together, whereas the network commonly known as the Internet and various biological networks are disassortative. See, Newman, M. E., “Assortative mixing in networks,” Physical Review Letters, 89(20):8701-8704 (2002).

### Diameter

The diameter, D, of a network is a global parameter defined as the longest of the shortest path, with the shortest path being the minimum path between two nodes. A measure related to the diameter is the average path length, <D>, which is the average over all the shortest paths. Those two parameters, however, require a very large amount of computing time to determine. A simple brute force algorithm on a sparse network where the shortest path between two nodes is determined by crawling will have an exponentially increasing complexity, described by the equation: k^{<D>}N². Another parameter, called the characteristic path length, L, has instead been introduced. This is the average of the shortest paths of randomly chosen pairs of nodes, selected a number of times so that this average converges. Even though this measure is not the diameter, it is characteristic of the network (Watts, D. J. and Strogatz, S. H., “Collective dynamics of ‘small-world’ networks,” Nature, 393(6684):440-442 (1998)).

### Identification of Critical Elements and Interactions

Application of graph metrics to the unbiased interactions network that has been refined by application of the maximum entropy principle results in a risk assessment map product that identifies the elements having critical importance to the development of the tested condition. In the medical/genetics context, the risk assessment map may be consulted to identify the key biomolecular constructs and interactions between biomolecular constructs that are critical to the development of the disease or syndrome that was the object condition of interest identified at the start of the method.

### Scoring of Data Elements for Criticality in Assessment of Risk

For each element of the map, a criticality score is computed that aggregates the result of each of the metrics applied. The criticality score is computed using un-weighted, function-designed (mathematically), or custom-weighted linear combinations of the results from single metrics. In specific cases, nonlinear combination can also be considered. Choice of either method to compute the criticality score will be dependent on the importance each metric score has relative to each of the other scores. Unweighted scoring is appropriate in cases where all metrics are considered equivalent (of equal weight).

The operation of the method of the present invention will now be illustrated in the following working examples, which are provided by way of illustration and not for purposes of limitation.

### EXAMPLES

### Example I: Assessment of Risk of Developing Arteriosclerosis

We produced a Risk Assessment Map product permitting evaluation of individuals' risk for developing arteriosclerosis with a low degree of bias and identification of the proteins and protein interactions that are of critical importance to the risk of developing arteriosclerosis.

(a) Extraction of Associated SNPs

We first compiled a database of reported single nucleotide polymorphisms (SNPs) associated with arteriosclerosis. We compiled our initial Associated SNP Database by extracting SNP identifiers from the Genome-Wide Association Studies (or GWAS) Catalog, which is a database collecting genotyping and analysis data on >100,000 SNPs without regard to gene locus or gene content from published peer-reviewed medical and scientific journal articles and science news reports. SNP information was selected as a starting point because it was a data-rich collection providing a great deal of publicly available information relevant to arteriosclerosis. The GWAS Catalog is co-curated by the National Human Genome Research Institute (NHGRI) of the National Institutes of Health (NIH) and the European Molecular Biology Laboratory-European Bioinformatics Institute (EMBL-EBI). It was accessed online at http://www.ebi.ac.uk/gwas. The data compiled in the GWAS Catalog is organized into 33 fields, and we extracted the standardized SNP identifier for any SNP associated with arteriosclerosis. This was tabulated in an Excel data set designated Arteriosclerosis SNP/Data Set 1. This data set contained a listing of 193 SNP identifiers, for example:

SNP

rs2059238
rs17132261
rs10911021
rs660240
rs10199768
etc.
. . .

(b) SNP Locus and Exclusion Based on Gene Proximity

The DNA locus of the SNPs identified from the GWAS Catalog was determined with reference to the current human genome sequence (Build #18 at NCBI36 repository). In this example, SNPs were eliminated from the table if their locus was more than 20 kilobases (20 kb) away from a gene. This exclusion step yielded a table of arteriosclerosis-associated SNPs linked with the corresponding gene and gene product, for example:

| SNP | Gene | Protein |
|---|---|---|
| rs2059238 | WWOX | WWOX |
| rs17132261 | SLC25A46 | SLC25A46 |
| rs10911021 | GLUL, ZNF648 | GLUL, ZNF648 |
| rs660240 | CELSR2 | CELSR2 |
| rs10199768 | APOB | APOB |
| etc. | | |
| . . . | | |

This data set was designated Arteriosclerosis SNP Proteins/Data Set 2.

The selection of the 20-kilobase proximity exclusion criterion is not critical. Because the databases at EMBL-EBI and scientific publications use different criteria to determine a gene locus and whether a SNP is located within a gene, selection of an expanded segment with respect to the reported locus of the gene ensured inclusion of gene-related SNPs and ensured consistency across data sources. The 20 kb proximity exclusion is a convenient exclusion factor to employ, as it is compatible with any mass data set including sequencing information. Alternative exclusion factors may be used, besides the obvious alternative of expanding or contracting the 20-kb threshold (e.g., expanding to 30 kb or contracting to 10 kb). One example of an alternative exclusion factor would be spatial colocalization, in which two features (e.g., SNPs and genes) must reside within a selected proximity in 3D space in order to be retained.

The elimination of SNPs located in faraway non-coding regions (outside the exclusion limit) was based on an assumption that such SNPs would have no effect or no recognized effect on the expression of any gene product or post-expression protein-protein interactions. This exclusion also was based on inclusion of only genes that have a known protein product; putative genes, for which there are no known transcribed proteins, were removed from the analysis.

(c) Retrieval of Protein-Protein Interaction Data for the SNP-Proximal Genes

For each of the identified proteins encoded by genes containing arteriosclerosis-associated SNPs or having SNPs within the inclusion margin (here, 20 kb), identification of other proteins with which it interacts was determined using the STRING and KEGG databases.

The STRING database of protein-protein interactions, is curated by the Swiss Institute of Bioinformatics (SIB), the Novo Nordisk Foundation Center for Protein Research (CPR), and the European Molecular Biology Laboratory (EMBL). STRING is a database of known and predicted protein interactions including direct (physical) and indirect (functional) associations, derived from four sources—genomic context, high-throughput experiments, conserved coexpression, and interactions reported in the scientific literature. The database was accessed at http://string-db.org.

The KEGG (Kyoto Encyclopedia of Genes and Genomes) database of genetic and molecular pathways integrates genomic, chemical and systemic functional information. Catalogs of genes from fully sequenced genomes are linked to systemic functions of the cell, the organism and the ecosystem. See, Kanehisa, M., “Toward pathway engineering: a new database of genetic and molecular pathways,” Science & Technology Japan, 59:34-38 (1996). The KEGG database resource is curated by Kanehisa Laboratories and was accessed at http://www.genome.jp/kegg.

Retrieval of protein interaction data proceeds for each protein in the Arteriosclerosis SNP Proteins/Data Set 2 and compiled all documented interactions, per protein. For example, the APOB protein, included in Data Set 2 is tagged as 9606.ENSP00000233242 in the STRING database, and that protein interacts with 1522 other proteins. These are first-degree interacting proteins.

| | | interaction |
|---|---|---|
| Interacting Protein | Primary Protein | scores |
| 9606.ENSP00000003084 | 9606.ENSP00000233242 | 0 0 0 0 0 0 |
| | | 224 224 |
| 9606.ENSP00000011653 | 9606.ENSP00000233242 | 0 0 0 0 0 0 |
| | | 215 215 |
| 9606.ENSP00000037502 | 9606.ENSP00000233242 | 0 0 0 0 0 0 |
| | | 226 226 |
| 9606.ENSP00000039007 | 9606.ENSP00000233242 | 0 0 0 368 0 0 |
| | | 228 479 |
| . . . | | |

The STRING database includes eight additional fields (i.e., neighborhood, fusion, co-occurrence, co-expression, experimental, database, text mining, and combined score), and sample values are shown for the protein interaction pairs above under the heading “interaction scores”. These fields contain confidence-level scores assigned by the database curators based on the nature of the interaction of the two proteins as derived from the data sources. We ignored these data and utilized only the fact of the protein-protein interaction pairing of the Primary Protein and the Interacting Protein.

After this first-degree interaction, we identified second-degree protein interactions, illustrated by the data listing below:

| Primary Interaction | |
|---|---|
| 9606.ENSP00000001008 | 9606.ENSP00000003084 |
| | 2nd Degree Interactions |
| 1st Degree Interaction | (from 9606.ENSP00000001008) |
| 9606.ENSP00000003084 | 9606.ENSP00000005558 |
| 9606.ENSP00000003084 | 9606.ENSP00000009180 |
| 9606.ENSP00000003084 | 9606.ENSP00000011292 |
| . . . | |

The STRING database lists only first-degree protein interactions, but from the first-degree interaction data, listings of second-degree interactions, then third-degree interactions, fourth-degree, etc., could be iteratively derived, until all the interactions between proteins listed in Data Set 2 had been compiled. Second- and higher-degree interactions are obtained by iteratively searching the database of first-degree interactions for each new protein new found at the previous iteration. The types of interaction are illustrated in FIG. 2, which diagrams protein-protein interactions among hypothetical proteins A, B, C, D, and E. The lines connecting some of the proteins represent protein-protein interactions. First-degree protein interactions are seen to exist between proteins A and B, proteins B and C, proteins B and D, and proteins C and D. Protein E does not have any known interaction with any of the other proteins in this set. Second-degree interactions are shown between proteins A and C, and between proteins A and D. There is also a second-degree interaction between proteins B and C (through D). A third-degree interaction is illustrated between proteins A and D (through B and C). The process is repeated until all interactions are found within one connected cluster of proteins or no additional new interactions are found.

Protein interactions per protein were added from the KEGG database, following the same process used with the STRING database. KEGG includes metabolic pathway data that is not available in STRING.

Each database uses a different nomenclature to refer to a protein, therefore hash tables (data element linker tables) were maintained to ensure proper access and use of these databases. Interrogation of the protein interaction databases proceeds until no further interactions per protein were found or until the found interactions accounted for all proteins in the original data set (here, Arteriosclerosis SNP Proteins/Data Set 2), indicating that the data set of proteins defines a cluster. The resultant data set including >11,000 protein-protein interactions was designated Arteriosclerosis Protein Interactions/Data Set 3.

(d) Construction of Adjacency Matrix from Protein Interactions Data

After completion of the Arteriosclerosis Protein Interaction/Data Set 3, an adjacency matrix was created using all the retrieved protein-protein interaction data from Data Set 3. In this matrix, each row and column represent proteins contained in the data set, and values in the matrix represent the interaction, or lack thereof, between the proteins. This matrix, which contains all known or expected interactions between the previously identified arteriosclerosis-related, SNP-containing proteins, defines the universe of possible protein-protein interactions relevant to the test condition (i.e., arteriosclerosis in this case).

An adjacency matrix for the protein interaction network illustrated in FIG. 2 appears below:

| | A | B | C | D | E |
|---|---|---|---|---|---|
| A | 0 | 1 | 0 | 0 | 0 |
| B | 1 | 0 | 1 | 1 | 0 |
| C | 0 | 1 | 0 | 1 | 0 |
| D | 0 | 1 | 1 | 0 | 0 |
| E | 0 | 0 | 0 | 0 | 0 |

As shown in the matrix above for hypothetical proteins A, B, C, D, and E, having a network of interactions as shown in FIG. 2, the absence of any direct interaction is scored as zero (0) and a first-degree protein-protein interaction is scored as one (1). The proteins are not regarded as interacting with themselves, so the matrix cells (A,A), (B,B), (C,C), (D,D), and (E,E) all have zero scores. Where two proteins have a known interaction, e.g., (A,B), (B,C), (C,D), etc. (see FIG. 2), the matrix cell has a score of one.

In the matrix created from Arteriosclerosis Protein Interaction/Data Set 3, there were 607 proteins and a total of 11,678 first-degree interactions. The resultant matrix data set was designated Arteriosclerosis Adjacency Matrix/Data Set 4.

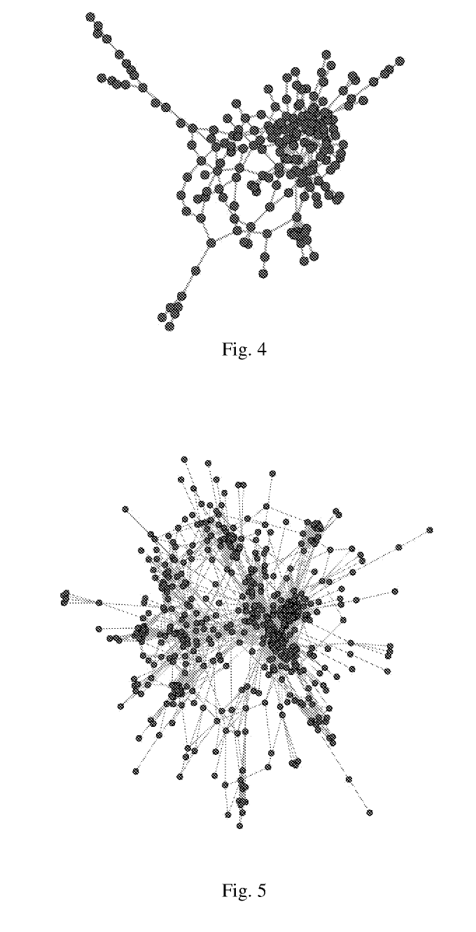

After creation of the Arteriosclerosis Adjacency Matrix/Data Set 4, further steps were performed on the data which were designed to reduce uncertainty in the interpretation of the interaction data. The matrix Data Set 4 may be advantageously visualized at this point by generating a graphic map. We generated protein interaction matrix maps using the open source Program R (R Development Core Team, R: A language and environment for statistical computing (R Foundation for Statistical Computing, Vienna, Austria 2008). ISBN 3-900051-07-0) to plot matrices of increasing size filled with protein interaction data from Data Set 4. Referring to FIG. 3, a matrix map was created from a 10×10 matrix using a random selection of 10 proteins and their interactions from Data Set 4. Referring to FIG. 4, a matrix map was created from a 100×100 matrix using 100 proteins and their interactions from Data Set 4. Finally, referring to FIG. 5, a map was created for a 1000×1000 matrix using 1000 proteins and their interactions from Data Set 4. The series of FIGS. 3, 4 and 5 illustrates the gain in matrix complexity from considering data sets of greater and greater size. As illustrated in Table 1, below, the complexity of analysis of interactions between proteins increases exponentially as more proteins are considered.

TABLE 1
Increase in complexity of protein interaction
networks with number of proteins analyzed

| | Number of Networks, | Total Number of |
|---|---|---|
| | assuming only one | Possible Networks, |
| Number of | protein/protein interaction | considering N |
| Proteins | per network | proteins |
| Analyzed N | N(N − 1)/2 | 2^{N(N-1)/2} |
| 3 | 3 | 8 |
| 4 | 6 | 64 |
| 5 | 10 | 1024 |
| 6 | 15 | 32,768 |
| 10 | 45 | 3.5 × 10¹³ |
| 45 | 990 | 10²⁹⁸ |
| 20,000 (number | 199,990,000 | (incomprehensibly |
| of proteins in | | large number) |
| a human cell) | | |

In a given population of proteins, each protein may have interactions with one or more proteins in the population, and the set of protein-protein interactions defined by one protein and all the other proteins of the population it interacts with is termed a network. The interaction may be physical, as where one protein binds to another protein, or may be functional, as when two proteins are co-expressed under given conditions. In the group of hypothetical proteins diagrammed in FIG. 2, a protein interaction network is shown by the interactions of proteins A, B, C, and D with one another. Protein E, which has no known interactions with any other protein, is not part of a protein interaction network. In the present example, if protein E was encoded by a gene containing or within 20 kb of an arteriosclerosis associated SNP, protein E would be included in Arteriosclerosis SNP/Data Set 1 and Arteriosclerosis SNP Proteins/Data Set 2, however the lack of any reported or expected interaction of protein E with any other protein would result in its being eliminated from Arteriosclerosis Protein Interactions/Data Set 3 and Arteriosclerosis Adjacency Matrix/Data Set 4.

If a set of N proteins is considered, and only pairwise protein interactions (i.e., first-degree interactions) are considered, then the total number of possible protein interaction networks is N×(N−1)÷2. Thus, in a set of six proteins, considering only single protein interactions, a total of fifteen protein interaction networks is possible. However, since proteins typically interact with a number of other proteins, if all the possible interaction networks are considered, i.e., wherein each protein in the set of N proteins interacts with zero up to all the other proteins (N−1) in the set, then the total number of possible protein interaction networks is 2^{N(N-1)/2}. Thus, in a set of six proteins, wherein the possible interactions of each protein is zero interactions up to five interactions, all possibilities for protein interaction networks amounts to 2¹⁵, or 32,768 (see, Table 1, supra).

In reality, a given protein typically has reported interactions with many other proteins; in fact, the number of interactions for one protein may number in the hundreds or thousands, as the example of protein APOB, mentioned above, shows—APOB participates in 1522 different reported protein-protein interactions—however, more typically, the majority of protein interactions per protein is from 4 to 20 other proteins. Even so, it can be appreciated that even if only a limited set of possibly relevant proteins is considered, the analysis of all potential interaction networks becomes impossible. For example, there are 33,554,432 possible networks when only considering 10 proteins and 25 known interactions, and recognizing that a number of these interactions either will not be relevant in a given cell type or will not be active during a given cellular process, the problem of extracting relevant interactions for consideration becomes daunting. This calculation of mathematically possible interaction networks does not describe a realistic population for analysis, when it is considered that only a small fraction of possible protein-protein interactions are chemically probable, and only a fraction of the chemically probable interactions will be biologically relevant. The Data Set 4 data set is extracted from compilations of experimentally confirmed protein-protein interactions and interactions reported in the peer-reviewed scientific literature, and accordingly the data set does not include protein-protein interaction networks for analysis that are completely unknown or that are completely speculative.

In view of the hyperbolic increase in the complexity of analysis of multiple proteins associated with a particular disease or syndrome, it becomes imperative in performing the analytical method of the present invention that the analysis of protein-protein interaction data be performed with the assistance of computer power. It is only by use of the multiplex calculation capability of computers that analysis of data sets listing more than, e.g., ten proteins, can be accomplished in a period of time to make the analysis practical and useful. Moreover, the required computing capacity increases with the number of proteins. For example, with commercially available personal computing capacity, protein data sets of about 1000 members can be analyzed according to the presently described method in less than a day. For protein data sets with higher orders of magnitude, dedicated institutional capacity computers (e.g., supercomputers, server farms, data centers) are necessary to obtain results within the same timeframe.

(e) Reduction of Uncertainty in Protein Interactions Matrix by Maximizing Entropy

The compilation of Arteriosclerosis Adjacency Matrix/Data Set 4 provided a universe of protein interaction networks having potential relevance to arteriosclerosis. Further processing of this data set was necessary to focus on the data that have the most relevance and are most reliable with respect to detection and treatment of arteriosclerosis and to eliminate bias and uncertainty from the data set. We adapted the maximum entropy method to minimize uncertainty from the Data Set 4 data set.

The maximum entropy method is used in various fields to reconstruct data models from imperfect or insufficient data. An example is gravitational lensing in astrophysics, where maximizing entropy allows reconstruction of images of distant astral bodies by correcting light data distorted by the gravitational fields of intervening objects such as galaxies. Where several images of a light emitting body fit the light data received by the earthbound observer, maximizing entropy ensures that the reconstructed image is the most probable image, given the data.

In a field relying on genetic, protein expression, and protein interaction data, we realized a similar problem existed of discriminating between many possible solutions fitting the available data. We used maximization of entropy to identify the protein interaction networks having the highest probability of relevance to the development of arteriosclerosis. We employed a Monte Carlo method to generate a series of relative entropy calculations using the protein interaction data in the data set (Data Set 4), each determining whether removal of one interaction at random from the data set increased or decreased entropy. Where removal of a particular interaction led to an increase in overall entropy, the interaction datapoint was returned to the data set; if removal of a particular interaction led to a decrease in the overall entropy, the interaction datapoint was left out of the data set as representing an interaction tending to bias the relationship of the data of Data Set 4 to accurate interpretation of arteriosclerosis data. By plotting each new entropy calculation according to the Lagrangian function Q=λS−χ², where S is entropy, χ² is error, and λ is a Lagrangian multiplier, the algorithm converges on a peak of maximum entropy, and the data set of protein-protein interactions taken at that peak represents the interactions having the highest probability of relevance to the development of arteriosclerosis. This data set was designated as Arteriosclerosis Roadmap/Data Set 5. It is a roadmap in the sense of having organized undifferentiated proteins and protein-protein interactions into a compilation of proteins and interactions of high relative importance, without unintentional bias. This is akin to a listing of topographical locations and connecting roads into an organized data set (roadmap) based on the relative importance for navigation to reach a desired destination, with uncertainty as to the importance of a given location or road eliminated. In biochemical terms, features that enhance or limit interactions, such as enzymes, promoter regions, 3D configurations, and the like, are akin to topographical features that affect the significance of location points on a map. This step, in other words, is a process for finding the distribution of protein interactions where probability of critical impact on arteriosclerosis is at a maximum, and where error/uncertainty/bias in the analytical data is minimized. The distribution of the data that maximizes the entropy gives the solution that contains the least bias.

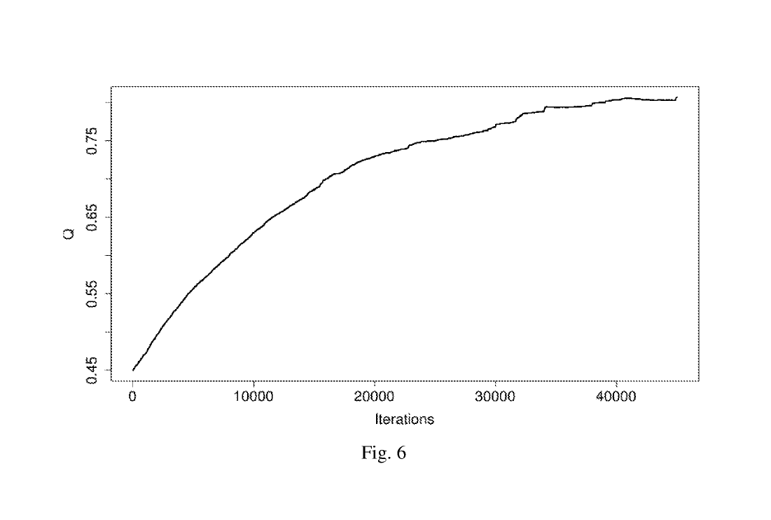

This process is carried out until the change in entropy plateaus, and elimination of individual elements does not lead to significant reductions in entropy. Referring to FIG. 6, the change in Q value is plotted as a function of the number of relative entropy calculations performed. It is seen that the entropy level plateaus, allowing the practitioner to stop the process when the change in entropy of the data set does not change significantly with further iterative calculations. As a practical matter, the process is typically stopped when the change in Q is no more than 1%-2% over a fixed number of iterations, such as 1,000, the less change occurring over the greater number of iterations indicating that a maximum has been reached. For example, <2% change in Q over 5,000 iterations, or more preferably over 10,000 iterations would be a stronger indication that maximum entropy has been reached. In FIG. 6, such a plateau was reached at around 40,000 iterations. Computer power and computer time can be limiting factors in this step, but it is most advantageous to carry out the maximization of entropy process until such a plateau is reached, so that the bias in the data set is minimized. It will be understood that in such a process, the maximization of entropy can be calculated forever, but for the purposes of completing this step of the method of the invention, “maximum entropy” is reached when the change in entropy ceases to show significant change (e.g., >2%) over a large number of calculations (e.g., >1000). The object of this step is to eliminate as much bias or uncertainty from the data set; therefore, ending the process before the rate of change in entropy reaches an apparent maximum leaves uncertainty in the data set.

(f) Application of Quantitative Metrics to Reveal Criticality of Unbiased Data

The data obtained in Arteriosclerosis Roadmap/Data Set 5 was refined further by application of quantitative metrics to determine quality of associations between each element of the Data Set 5 data set, based on its functionality, its relationship to other elements, and its criticality in the biological system(s) it is a part of. For the Data Set 5, we computed quantitative metrics on each data element to create a metric matrix, M, where elements for protein i are the clustering coefficient (Cᵢ), degree of connectivity (kᵢ), and centrality (Bᵢ). A sample fragment of the matrix M thus appeared as follows:

M

| Protein i | WWOX | SLC25A46 | GLUL | CELSR2 | APOB | . . . |
|---|---|---|---|---|---|---|
| clustering | 0.33 | N/A | N/A | N/A | 0.19 | . . . |
| coefficient | | | | | | |
| (Cᵢ) | | | | | | |
| centrality | 0.55 | N/A | N/A | N/A | 2.28 | . . . |
| (Bᵢ) | | | | | | |
| degree of | 3 | N/A | N/A | N/A | 15 | . . . |
| connectivity | | | | | | |
| (kᵢ) | | | | | | |

N/A = Not Applicable, because this protein was removed from the data set during reduction of uncertainty, conducted in step (e). These proteins (e.g., GLUL, CELSR2) contributed to a reduction of entropy (increase of uncertainty).

The metric matrix provided a plurality of values for each data element of Data Set 5 that permits the elements to be distinguished from one another in terms of structural and functional relationships between proteins of an interaction network. The data set was designated Quantitative Metric Matrix/Data Set 6.

(g) Scoring of Metric Matrix Data Elements to Provide a Risk Assessment Product

With the values ascribed to each protein interaction obtained by application of quantitative metrics, it was possible to compute the risk value, R, for each protein of the Arteriosclerosis Roadmap, using a linear combination of the metrics, such as R=MW^{T}, where M is a matrix containing the values, per protein, for each of the calculated metrics and W^{T} is a transposed matrix of the respective weight associated with each of the metrics. For example, the weight in the matrix reflects that higher betweenness values are more critical than lower. The proteins and protein-protein interactions were ordered according to their risk scores, which yielded a hierarchical listing of 574 proteins involved in the development of arteriosclerosis. A fragment of the listing appeared as follows, showing the proteins determined by our method to be most important to the development of arteriosclerosis. Shown in the table below are the ten highest risk-associated proteins and their risk scores, ten proteins from the middle rank of the listing, and the ten lowest risk-associated proteins.

Risk Score (R)

| Protein | R |
|---|---|
| ADCY9 | 1432 |
| ERCC4 | 1383 |
| FGB | 1259 |
| LPL | 1250 |
| AK1 | 1184 |
| YKT6 | 1172 |
| EIF3H | 1113 |
| FGA | 1109 |
| ABCA1 | 1092 |
| APOB | 1065 |
| . . . | . . . |
| GJA1 | 688 |
| GPN1 | 679 |
| AIM1 | 655 |
| NCAM1 | 628 |
| WWOX | 625 |
| LRAT | 543 |
| BACE1 | 523 |
| PROCR | 516 |
| LRIG1 | 503 |
| ATP6V1C2 | 501 |
| . . . | . . . |
| GCKR | 386 |
| EDC4 | 374 |
| TAGLN | 373 |
| CETP | 318 |
| FADS1 | 310 |
| WDR1 | 286 |
| FBLIM1 | 260 |
| TFAP2B | 133 |
| GALNT2 | 86 |
| GRID1 | 77 |

The risk scoring provided a Risk Assessment Database product, wherein a risk score was ascribed to all proteins in the Arteriosclerosis Roadmap/Data Set 5, based on structural and functional features of the network. This results in a risk map with which biological profiles of individuals may be evaluated. Such a predictive tool produced by this invention is far superior to diagnostic estimation of probabilities of developing a disease, in this case arteriosclerosis, based on historical correlations between one or more genetic polymorphisms and development of the disease because bias in the probability of the role of the disease has been minimized, and the data have been focused to increase the accuracy of interpretation (i.e., to identify the criticality of the role of a given protein, protein interaction, or pathway).

The risk map is a powerful and accurate tool, however it will also be understood that the scores computed are subject to change as more and more research is performed and new data are added to the genomics, proteomics, metabolomics, and other “omics” databases that are interrogated according to the present invention. For this reason, the accuracy of the risk map product may be improved over time by repeating the process to include consideration of subsequently added research results and reports.

### Example II: Assessment of Individual's Risk of Development of Arteriosclerosis

The Risk Assessment Database product from Example I was used to assess the predisposition of two hypothetical individuals to develop arteriosclerosis.

A hypothetical sample population was created by randomly generating SNP profiles of 1000 hypothetical individuals based on the 574 proteins identified in Example I as highly relevant to arteriosclerosis. For each protein, one of the two SNP variants reported in the GWAS Catalog was randomly assigned, i.e., so that for each of the 574 proteins, the individual would harbor the variant associated with arteriosclerosis or a variant not associated (or less associated) with development of arteriosclerosis. The 1000 profiles were scored using the Risk Assessment Database product produced in Example I, and plotting the scores produced a normal bell curve. This plot was used as a standard curve against which to compare two exemplar profiles, one for a hypothetical Subject A and one for a hypothetical Subject B.

The profile of a hypothetical Subject A was created by first randomly ascribing disease-associated variants to the set of the 574 proteins. Then a selection criterion was set regarding the ten highest ranking disease associated proteins of the 574 which forced more than 50% of the proteins to exhibit the disease-associated variant. This presumably created a Subject A having a high risk for development of arteriosclerosis.

The profile of a hypothetical subject B was composed by randomly ascribing either the disease-associated variant or the non-disease-associated variant for each of the 574 proteins.

The profiles of Subject A and Subject B were then compared against the Risk Assessment Database product created in Example I.

Gene products were identified for each of the SNPs for Subject A and Subject B. The individual susceptibility of Subject A and Subject B were assessed by interrogating the risk map with the hypothetical profiles composed as described above. Individual risk was assessed according to the function Rₘ=RP=ax+by+cz+ . . . , where R is the risk matrix value defined above, P is the SNP profile of the individual, the variables a, b, c, etc. are quantitative measures of criticality for each protein from the Risk Assessment Database, and x, y, z, etc. are values ascribed for each of the proteins being assessed from the individual subject profiles, to contrast with the risk assessment roadmap.

Subject A had a risk score of 945/1000, indicating very high probability of developing arteriosclerosis; Subject B had a risk score of 175/1000, indicating a low risk of developing arteriosclerosis. Analysis of the proteomic data for Subject A showed a high number of disease-associated SNPs in highly ranked proteins of the R data product, whereas the SNP profile of hypothetical Subject B showed a low proportion of disease-associated SNPs in proteins listed in the Risk Assessment Database produced in Example I.

The results from these models indicate that the risk assessment tool created according to the invention easily distinguishes between a high risk arteriosclerosis patient and a healthy normal hybrid profile.

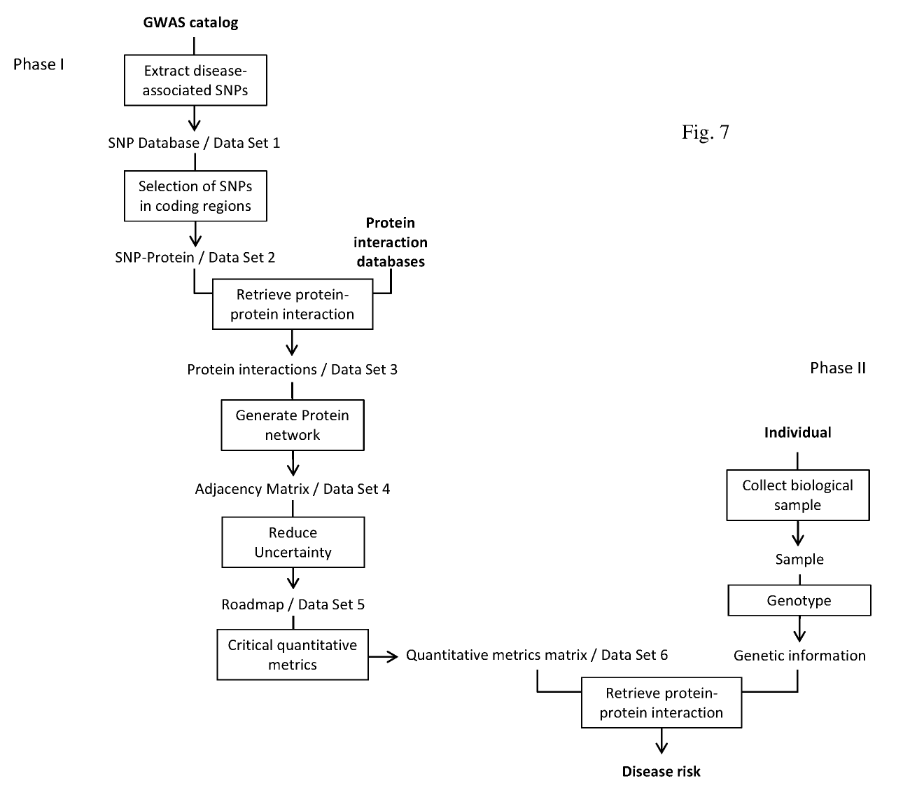

The steps of Examples I and II are illustrated schematically in FIG. 7.

### Example III: Assessment of Risk of Developing Autism

Following the general methodology illustrated in Example I, a Risk Assessment Database product is generated for assessment of risk for development of Autism Spectrum Disorder, a complex early childhood onset disease.

Autism Spectrum Disorder is a general term for a wide range of complex social communication and behavioral interaction disorders with genetic and environmental confounding factors associated with the disorder, as reported in the literature. These disorders are characterized, in varying degrees, by difficulties in social interaction, difficulties in verbal and nonverbal communication, and repetitive behaviors. Autism can be associated with intellectual disability, difficulties in motor coordination and attention, and physical health issues such as sleep and gastrointestinal disturbances. Some persons diagnosed with autism excel in visual skills, music, math and art.

Autism appears to have its roots in very early brain development, and the most obvious signs of autism tend to emerge between 2 and 3 years of age. Early diagnosis and early intervention with behavioral therapies can improve outcomes, and therefore a more accurate risk assessment tool would be helpful in identifying infants at risk for autism and would lead to more effective treatment.

The GWAS Catalog is screened for genetic variants associated with autism, generating a listing of gene loci of interest with regard to the test condition (autism). Genetic loci are linked with expressed gene products by consultation of the human genome sequence, and the gene products are used to interrogate the STRING and KEGG data collections to collate protein-protein interactions and metabolic pathways implicated. Next, an adjacency matrix is constructed from the interactions and pathways data to yield a data set representing the universe of possible protein-protein interactions to be considered as relevant to the test condition. Bias is minimized in the resulting data set by maximizing entropy, calculated in the same manner as in Example I. Following maximization of entropy, which eliminates many proteins from the previous data set, a series of quantitative metrics is applied to reveal criticality in the retained data, to yield a metric matrix. Each element in the metric matrix is assigned a risk value using an unweighted linear combination of the metrics scores, which results in a risk assessment database containing members that can be ranked according to their risk values. This database can be used as a risk assessment tool against which individual genome profiles may be compared to gauge risk of developing autism.

The risk assessment database makes it possible to make use of very early samples of genetic information, e.g., obtained from a newborn, in order to make an early assessment of autism risk. In individuals showing a genetic profile corresponding to high autism risk when compared with the risk assessment database, heightened attention to detecting the first signs and indications of neurodevelopmental problems, and earliest possible behavioral intervention programs, may be instituted.

All of the publications and documents cited above are incorporated herein by reference.

1. A method for production of a risk assessment data map comprising the following steps:
(a) selecting from a mass data collection a set of data elements having an association with a condition of interest;
(b) constructing an integrated multidimensional network from the initial selected set of data elements by collecting data, for each element, relating to interactions with any other element;
(c) sorting the information from the integrated multidimensional network using mathematical functions to eliminate elements of lesser relevance to the condition of interest, by minimization of bias; and
(d) applying quantitative metrics to the retained elements of the multidimensional network to create a data map that gives relative weight to the retained elements and element interactions, identifying the criticality of each element and interaction with respect to the condition of interest.

2. A method for assessing the risk of realizing a condition of interest from an individual set of elements comprising:
(a) comparing said individual set of elements to a risk assessment data map according to claim 1, and
(b) assessing the degree of matching of individual elements with corresponding elements of the risk assessment data map that is associated with the condition of interest.

3. A method for producing a risk assessment map for a physiological condition comprising the steps:
(a) selecting a set of biomolecular constructs associated with a physiological condition to be diagnosed or treated;
(b) constructing an integrated multidimensional network detailing biophysical and biochemical properties and interactions of the selected biomolecular constructs;
(c) tuning the amount of information to be retained in the multidimensional network using mathematical functions to ensure minimization of bias to yield an unbiased multidimensional network; and
(d) computing the criticality of each biomolecular construct in the resulting unbiased multidimensional network by application of graph metrics, to yield a risk assessment map detailing the biomolecular constructs and interactions between biomolecular constructs that are critical to development of the physiological condition.

4. A method for assessing the susceptibility of an individual or group of individuals to developing a physiological condition of interest, the method comprising:
(a) preparing a risk assessment map by the method according to claim 3;
(b) establishing a profile for an individual, from a biological sample obtained from the individual, by identifying the set of biomolecular constructs corresponding to the set selected in the preparation of said risk assessment map;
(c) computing the risk of the individual to develop the physiological condition of interest by mapping the profile of step (b) to said risk assessment map and assessing the differences between the profile and the biomolecular constructs and interactions between biomolecular constructs that are critical to development of the physiological condition of interest, as detailed in said risk assessment map.

5. The method of claim 3, wherein said biomolecular constructs are selected from genes, genetic polymorphisms, transcribing elements of genomic material, proteins, genetic mutations, protein isoforms, and combinations thereof.

6. The method of claim 5, wherein said biomolecular constructs are genetic polymorphisms.

7. The method of claim 6, wherein said biomolecular constructs are single nucleotide polymorphisms (SNPs).

8. The method of claim 3, wherein said physiological condition is a disease or syndrome.

9. The method of claim 8, wherein said selecting step (a) is carried out by compiling a database of biomolecular construct elements associated with said physiological condition by interrogating one or more mass data collections.

10. The method of claim 9, wherein said mass data collections include one or more omics data repositories.

11. The method of claim 10, wherein said tuning step (c) is carried out by maximizing entropy of the data of the multidimensional network.

12. The method of claim 11, wherein said computing step (d) is carried out by applying to the unbiased multidimensional network resulting from step (c) a series of graph metrics including degree of connectivity, degree of clustering, assortativity, and network diameter.

13. A diagnostic method for determining susceptibility of an individual to develop arteriosclerosis comprising monitoring two or more proteins selected from the group consisting of:

| | | | | |
|---|---|---|---|---|
| ADCY9 | EIF3H | AIM1 | LRIG1 | FADS1 |
| ERCC4 | FGA | NCAM1 | ATP6V1C2 | WDR1 |
| FGB | ABCA1 | WWOX | GCKR | FBLIM1 |
| LPL | APOB | LRAT | EDC4 | TFAP2B |
| AK1 | GJA1 | BACE1 | TAGLN | GALNT2 |
| YKT6 | GPN1 | PROCR | CETP | GRID1 |

to detect dysregulation of the proteins in said individual.

14. A method of decreasing the susceptibility of an individual to developing arteriosclerosis comprising administering to said individual an amount of an agent effective to at least partially correct dysregulation in an individual of a protein selected from the group consisting of:

| | | | | |
|---|---|---|---|---|
| ADCY9 | EIF3H | AIM1 | LRIG1 | FADS1 |
| ERCC4 | FGA | NCAM1 | ATP6V1C2 | WDR1 |
| FGB | ABCA1 | WWOX | GCKR | FBLIM1 |
| LPL | APOB | ERAT | EDC4 | TFAP2B |
| AK1 | GJA1 | BACE1 | TAGLN | GALNT2 |
| YKT6 | GPN1 | PROCR | CETP | GRID1 |
